(19) United States
(12) Patent Application Publication
(10) Pub. No.: US 2018/0055870 A1
(43) Pub. Date: Mar. 1, 2018

(54) VISCOSE-AMINO BODILY FLUIDS CONTRA

(71) Applicant: Jennifer Lee Powers, (US)

(72) Inventor: JENNIFER LEE POWERS, Seattle, WA (US)

(21) Appl. No.: 15/234,895

(22) Filed: Aug. 29, 2016

(51) Int. Cl. A61K 31/717; A61K 35/55; A61K 36/53; A61K 36/00

(52) U.S. Cl. CPC A61K 31/717; A61K 36/00; A61K 36/53; A61K 35/55

(57) ABSTRACT

The abstract of the disclosure is to give clear understanding of what Viscose-Amino Bodily Fluid CONTRA is and how it is a very readily available ingredient in manufacturing products and an ingredient on Pharmaceuticals and how it replaces the hard-to-find Human Tissue donations which was almost extincting a majority of products on the market today for non availability of supplies.

Viscose-Amino Bodily Fluid CONTRA is a Clearish-Grey gel fluid that has a watery gel like texture and much like a personal body lubricant such as K-Y gel, it has a slippery wet feel to it and is collected from a Human's sweat ports located in their pelvic area (one port on each side just off to the outer side of any human male or female's reproductive organs and is passed down usually through their reproductive orifices and exactly collected like Fluidity Alcohol and Beverage Beer Contro a different patent than mine, and with belts with containers in smalleau 'u and with tubes with inline sump pumps to collect the fluids after they have leaked out.

This product is made by altering to three things, Viscose the largest amount, A Flora Hyssop Ipsuu plant part and an Indonesian Bavo-Baverian Byline plant part and is the basic formula for Viscose Contra formulas that are previously patented and none of them registered with this formula exact. This formula with an extra ingredient, Rubber, had already been patented in my behalf and as I was involved with their creations and development and they were developed using my formula by the National Guard Legal Division and Military Tribunal located in Seattle, Wash. at 700 Stewart St., Courtroom #1 had registered the Rubber formula in my behalf and of which had stated for me to apply for a patent on my own for the basic formula that I am requesting patent on in this application today.

This is also makes available in the future this basic formula so that others may benefit from using my formula and so I request that I get 50% of all monetary for the future use of this formula. The basic formula is to be considered different from that of the Alcohol Beverage Contra as it must have a High content of Viscose in it and that is not a composition in beverages at least up to the start of this application but I do not believe that it would pose any health problems if they were to include it in any beverage production but it is not the purpose of this application.

Finally, while Viscose-Amino Bodily Fluid CONTA is able to be used widely in all manufacturing Sundries and all 8 categories of manufacturing products and goods, its largest benefit is that it is so easily produced, collected and able to be obtained easily and that it has replaced the formerly used body tissue donations which are extremely hard to get ahold of in large quantities that it almost extinted many products on the market today.

My written description of my product, is made and collected the identical way as a prior patented product, Alcohol beverages, to name one famous one prior patented claim. I list in Cross-Reference to Related Applications how mine is collected the same as Alcohol is collected and including produced within the human body and flowed out body sweat ports or glands located near a human's reproductive anatomy just off to the side of them and in the pelvic area and usually flowed out a human body from their reproductive orafices. The purpose of this Patent invention is all in the formula; the formula aids almost all manufacturing being produced on the entire planet as it has the same compositions of human enzymes and amines that work well in manufacturing and can replace what was always used as an ingredient—a hard to find human body tissue donation and for my invention—a readily produced, collected in abundancy and always able to be obtained.

The specific description of my already produced product (this product is already being HEAVILY used by major manufacturers today since I developed it in August, 2016 at home and at Lowes Home Centers where I work as a Night Stocker at store #0004 but of which I chose to patent it myself) lies entirely with the formula or should I say formula plus whatever the heredity alteration variations are of the human who produces it from their sweat glands and intentionally altering to variations of things such as different Flora and or Fauna to produce variations to my basic formula: which is the main topic of patent with the variations included that was minimally mentioned in original application as depending on what the human composition is at time of production. Most manufacturers would like to “Trademark” the various alterations and will probably want to request specific intentional alterations for their individual patented Trademarks so it is unique to their own stores but they will have to take that up with us, who will be doing the producing of the product itself on a separate issue and they will probably want to register the variations later. I am at least wanting Patent for the basic product and to let it be known that I can alter to various Flora or Fauna or objects to cause the further variations while producing my basic product.

My product itself's formula-1 extra ingredient that is unique to any other Patent, which comprises of 2 ingredients in Alcohol products and in combination with my unique ingredient make up my formula's basic formula and is required to be a Top Secret Trademark for all category manufacturing and also that I own rights to it and will only allow 50% (fifty percent) Royalty on any usage of my idea, product formula and derivative options for product for anyone else who will want to use it in the future.

### CROSS-REFERENCE TO RELATED APPLICATIONS

The cross-reference is to be made to prior applications and prior patents for Alcohol and Beverage patents for the Belts and apparatuses such as container holders, mini sump pumps and on hoses with flexible plastic inlet openings which collect bodily fluids from human body sweat duct glands (2 are located in Pelvic area 1 sweat port on right and left side just off of the reproductive anatomy and which flow out either Reproductive canals (for females) or out a Male's reproductive anatomy or openings and into plastic hoses with mini sump pumps which can collect large amounts of product in a short amount of time and direct the flow into a collector container, in regular sized or sometimes in smalleau and in an innuous or placed under garments and collected on a holding belt. This process is already been patented and is not the claimage in this Patent application but merely the same way in collecting the fluids the way that my product is obtained and with the 2 ingredients that cause the human fluidity and composition and in all applications of production, collection in containers and distribution the same way.
1. I have and have had prior Alcohol and Beverage licenses in the State of Washington or Oregon called Fluidity Alcohol Beer Beverage and also Sundries Production formerly listed under Pornagraphy but in the last 2.2 years it is listed under CONTRA Subdural Subturberiminal licenses; newest License is #1133684 Oregon valid from April 2016 until Apr. 8, 2018.
2. There is also another Patent Trademark for a formula, of which I am also the founder of that has 1 more ingredient that I use in this recipe/formula, which is Viscose and that is patented called Rubber Viscose Contra and it registered and applied through either the US Constitution military Tribunal National Guard Legal Dept. located in Seattle, Wash. 206-370-5237 phone number and of which I own rights to in either this name or an A.K.A. name under my confidential file in a CIA database #01123iccei and which also uses my basic formula with other ingredients such as Rubber and some Balsa at times. I do claim that I am the inventor of this basic formula here listed in detailed description.

The Manner in which my product is collected is the same as Fluidity Alcohol beverages are collected exactly but it is What my product formula's composition is with the unique altered to ingredient and described as which is why it is highly sought after and benefits manufacturing of goods that is the claim to this patent.

### DETAILED DESCRIPTION OF THE INVENTION

The detailed description of the Invention is all about what the formula is of it's consistancy and DNA composition which is what is different and unique to it's own rights to be registered and lay claims to ownership of and is the purpose of this Patent; to patent the formula of my product.

As stated earlier in Specification and Cross-Reference to Related Products, the way produced from a human as a fluid and collected is not what I am patenting as it is already a Trademark of Fluidity Alcohol Beverage products formerly listed under Pornography but now listed under CONTRA Subdural Subterberiminal and where I already have a license for production of Beer and Sundries products and am also using 2 ingredients of numerous compositions which are included in the prior formulas which are all Trademarked as Alcohol beverages or any other product that is made and on the market today. These 2 ingredients (or what I call variations to a persons composition for purposes of creating DNA compositions by altering Flora, Fauna or what have you, to alter to those ingredients to give unique formulas for product variations) are a major 2 variations as they are necessary for the fluidity and consistancy that is already sent in and written about in the original application.

I am not patenting either formulas listed in cross-reference of the 2 ingredients for Alcohol beverages by themselves nor 1 which is used for a newer Patent Trademark of similar recipe/formula but has Rubber as a main component but a recipe/formula that ONLY has 3 components/ingredients as the basic formula and what I call my own formula.

The Fluidity Alcohol beverage patented Trademark's 2 ingredients are necessary for humans to create a perfuse fluidity condition and the newer Patented formula for Rubber-Viscose, that a Court/tribunal located at 700 Stewart St. Courtroom #1, Seattle Wash. 98101 had registered in my behalf as I had founded it as well, has the other major ingredient of Viscose that is so necessary for my basic formula and can be more widely used than with the Rubber content as the prior patent. This basic formula and the base for all the variations I am claiming is also my own and is backed by both Courts located at the above mentioned court address and has its own uniqueness to any other patent registered that I am claiming here in this application for Patented Trademark.

My basic formula that I am registering here is as follows:
I, a Fledgling made of a person in good to excellent health and sound mind having altered to 2 ingredients that are already a known in the Alcohol Beverage world as a the necessary 2, which cause fluidity and consistancy, and which called Indonesian Bavo-Bavarian Byline (2-3 parts) and the Hosprecka of a Hissop Ipsuu (4-5 parts), both Flora derived.
The second step is the unique ingredient that makes this basic formula it's own unique formula, and that is adding Viscose to what I alter to which makes up the 3 ingredients to alter to for this formula.

1. A fluid produced same way as Fluidity Alcohol beverages and stored in containers made with 3 ingredients of Hipcine of Bavo-Bavarian 2-3 parts, Hissop Ipsuu Hosprecka of 4-5 parts and Viscose 5 parts and with appearance of a clear/greyish semi-Jellied viscose fluid non-odorous and displays on DNA machines as high percentage of Hu Humano-Amines en embigatoriee ae and I names Viscose-Amino Bodily Fluid-CONTRA.
